SEQUENCE LISTING (19 sequences)

SEQ ID NO: 1 - protein, 10 aa, Artificial Sequence
  exemplary hinge region which comprises cysteine residues
DKTHTCPPCP

SEQ ID NO: 2 - protein, 10 aa, Artificial Sequence
  exemplary hinge region which does not comprise cysteine residues
DKTHTAPPAP

SEQ ID NO: 3 - protein, 263 aa, Artificial Sequence
  exemplary fusion polypeptide of the invention comprising 4 hinge regions
DKTHTAPPAPSRDKTHTAPPAPVDDKTHTAPPAPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 372 aa, Artificial Sequence
  exemplary fusion polypeptide of the invention comprising 4 hinge regions
ATGVRAVPGNENSLEIEELARFAVDEHNKKENALLEFVRVVKAKEQMKNTDFALATMYYLTLEAKDGGKKKLYEAKVWVKDVQWYGTLHNFKELQEFKPVGDARSVDPWDKTHTAPPAPSRDKTHTAPPAPVDDKTHTAPPAPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 285 aa, Artificial Sequence
  exemplary fusion polypeptide of the invention comprising 4 hinge regions
DKTHTAPPAPSRDKTHTAPPAPVDDKTHTAPPAPRSDKTHTCPPCPAPELLGGPSVFLFPPKPKDTLMISRTPEVTCVVVDVSHEDPEVKFNWYVDGVEVHNAKTKPREEQYNSTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTCY

SEQ ID NO: 6 - protein, 394 aa, Artificial Sequence
  exemplary fusion polypeptide of the invention comprising 4 hinge regions
ATGVRAVPGNENSLEIEELARFAVDEHNKKENALLEFVRVVKAKEQMKNTDFALATMYYLTLEAKDGGKKKLYEAKVWVKDVQWYGTLHNFKELQEFKPVGDARSVDPWDKTHTAPPAPSRDKTHTAPPAPVDDKTHTAPPAPRSDKTHTCPPCPAPELLGGPSVFLFPPKPKDTLMISRTPEVTCVVVDVSHEDPEVKFNWYVDGVEVHNAKTKPREEQYNSTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTCY

SEQ ID NO: 7 - protein, 22 aa, Artificial Sequence
  example of two hinge regions with a short amino acid sequence between the hinge regions.
DKTHTCPPCPRSDKTHTCPPCP

SEQ ID NO: 8 - protein, 34 aa, Artificial Sequence
  example of three hinge regions with a short amino acid sequence between each of the hinge regions
DKTHTCPPCPVDDKTHTCPPCPRSDKTHTCPPCP

SEQ ID NO: 9 - protein, 46 aa, Artificial Sequence
  example of four hinge regions with a short amino acid sequence between each of the hinge regions
DKTHTCPPCPSRDKTHTCPPCPVDDKTHTCPPCPRSDKTHTCPPCP

SEQ ID NO: 10 - protein, 22 aa, Artificial Sequence
  example of two hinge regions with a short amino acid sequence between the hinge regions
DKTHTAPPAPRSDKTHTCPPCP

SEQ ID NO: 11 - protein, 34 aa, Artificial Sequence
  example of three hinge regions with a short amino acid sequence between each of the hinge regions
DKTHTAPPAPVDDKTHTAPPAPRSDKTHTCPPCP

SEQ ID NO: 12 - protein, 46 aa, Artificial Sequence
  example of four hinge regions with a short amino acid sequence between each of the hinge regions
DKTHTAPPAPSRDKTHTAPPAPVDDKTHTAPPAPRSDKTHTCPPCP

SEQ ID NO: 13 - protein, 4 aa, Artificial Sequence
  spacer sequence
LVLG

SEQ ID NO: 14 - protein, 249 aa, Artificial Sequence
  Reference fusion polypeptide that forms hexameric proteins (also designated “Hexa-Fc or HexaGard”)
DKTHTCPPCPAPELLGGPSVFLFPPKPKDTLMISRTPEVTCVVVDVSHEDPEVKFNWYVDGVEVHNAKTKPREEQYNSTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTCY

SEQ ID NO: 15 - DNA, 792 bp, Artificial Sequence
  DNA sequence of the amino acid sequence set out by SEQ ID NO 3
gacaaaactcacacagccccaccggccccatctagagacaaaactcacacagccccaccggccccagtcgacgacaaaactcacacagccccaccggccccaagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 16 - DNA, 858 bp, Artificial Sequence
  DNA sequence of the amino acid sequence set out by SEQ ID NO 5
gacaaaactcacacagccccaccggccccatctagagacaaaactcacacagccccaccggccccagtcgacgacaaaactcacacagccccaccggccccaagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtctgcctccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaattagtcctaggaccccccctgtacaacgtgtccctggtcatgtccgacacagctggcacctgctactga

SEQ ID NO: 17 - protein, 105 aa, Artificial Sequence
  Exemplary payload sequence - Zika virus EPIII domain
GMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQ

SEQ ID NO: 18 - protein, 105 aa, Artificial Sequence; identical to SEQ ID NO: 17
  Exemplary payload sequence - Dengue virus EPIII domain

SEQ ID NO: 19 - protein, 103 aa, Artificial Sequence
  Exemplary payload sequence - F1 adhiron
ATGVRAVPGNENSLEIEELARFAVDEHNKKENALLEFVRVVKAKEQMKNTDFALATMYYLTLEAKDGGKKKLYEAKVWVKDVQWYGTLHNFKELQEFKPVGDA

(19) United States
(12) Patent Application Publication
(10) Pub. No.: US 2021/0061887 A1
(43) Pub. Date: Mar. 4, 2021

(54) FUSION PROTEIN COMPRISING MULTIPLE HINGE SEQUENCES

(71) Applicant: LIVERPOOL SCHOOL OF TROPICAL MEDICINE, Liverpool (GB)

(72) Inventors: Richard Pleass, Liverpool (GB); Pat Blundell, Liverpool (GB)

(21) Appl. No.: 16/981,131

(22) PCT Filed: Mar. 15, 2019
(86) PCT No.: PCT/GB2019/050745

§ 371 Date: Sep. 15, 2020

(30) Foreign Application Priority Data
Mar. 16, 2018 (GB) 1804243.2

(51) Int. Cl. C07K 16/00

(52) U.S. Cl. CPC C07K 16/00; C07K 2319/33; C07K 2317/53; C07K 2317/52

(57) ABSTRACT

The invention relates to fusion polypeptides derived from IgG. The fusion polypeptides comprise a domain derived from an immunoglobulin heavy chain constant region; and a plurality of hinge regions derived from IgG hinge sequences. The invention also relates to nucleic acids encoding the fusion polypeptides, and to proteins comprising two fusion polypeptides of the invention. Also provided are a pharmaceutical composition comprising a fusion polypeptide and/or protein of the invention, and medical uses of the polypeptides, proteins, or compositions.

### FIELD OF THE INVENTION

The present invention relates to a fusion polypeptide comprising a domain derived from an immunoglobulin heavy chain constant region and a plurality of hinge regions derived from IgG hinge sequences. The invention also relates to a protein comprising two fusion polypeptides of the invention, and a pharmaceutical composition comprising the fusion polypeptide and/or the protein of the invention. Further, the invention relates to the use of the fusion polypeptide, the protein and/or the pharmaceutical composition as a medicament, and to a nucleic acid encoding the fusion polypeptide.

### BACKGROUND

Fc-based fusion proteins are composed of an immunoglobulin Fc domain that is directly linked to another molecule, such as a peptide, or other important therapeutic molecule. The fused or conjugated partner can be any proteinaceous or non-proteinaceous molecule of interest, e.g. a receptor or peptide that mops up disease-causing cytokines, a peptidic antigen for immunizing against a challenging pathogen, or a ‘bait’ protein to identify binding partners assembled in a protein microarray, or a small molecule.

Most frequently, the fused partners have significant therapeutic potential, and they are attached to an Fc-domain that gives the hybrids several beneficial biological and pharmacological properties. Perhaps most importantly, the presence of the Fc domain markedly increases the fused or conjugated molecule's plasma half-life, which prolongs therapeutic and/or vaccine activity, owing to its interaction with the salvage neonatal Fc-receptor (FcRn), and to slower renal clearance of larger molecules. The attached Fc domain also enables these molecules to interact with Fc-receptors (FcRs) found on immune cells, a feature that is particularly important for their use in oncological therapies and vaccines. From a biophysical perspective, the Fc domain folds independently and can improve the solubility and stability of the partner molecule both in vitro and in vivo, while from a technological viewpoint, the Fc region allows for easier and more cost-effective purification by protein-G/A affinity chromatography during manufacture.

The advantages of Fc-fusion drugs over other types of biopharmaceuticals have been extensively reviewed. In terms of market value, Fc-fusion proteins and monoclonal antibodies (mAbs) together account for 43% of all therapeutic proteins based on published sales in 2008, and examination of those in the clinical pipeline suggests that this market share is likely to rise (http://clinicaltrials.gov/). These figures do not include Fc-fusion proteins conjugated to non-proteinaceous molecules, for example small molecules.

Most Fc-fusions target receptor-ligand interactions, work either as antagonists to block receptor binding (e.g. etanercept, aflibercept, rilonacept, belatacept, abatacept) or as agonists to directly stimulate receptor function to reduce (e.g. alefacept), or increase immune activity (e.g. romiplostim). However, in their present configuration such Fc-fusions can only bind two target molecules and deliver them to a single cell-surface receptor.

Hexameric Fc-fusion proteins have been previously described. These proteins are able to bind twelve target molecules, thereby delivering the molecule with therapeutic interest in much greater quantity. However, these hexameric Fc-fusions show a complete lack of binding to all FcγRs investigated to date, and a marked reduction in binding to the FcRn, compared to hexamers expressed in the absence of the fusion partner. Although this may have distinct advantages in certain clinical applications, where enhanced ligand interactions are desirable in the absence of FcγRn and/or FcR binding (e.g. intravitreal injection of aflibercept to treat macular degeneration where interactions with the FcRn or FcγRs may not be desirable), for other applications the inability to engage these receptors may be disadvantageous (e.g. in maintaining a long half-life for etanercept in the circulation).

Accordingly, Fc-fusion proteins, which are able to bind more than two target molecules, yet retain the ability to bind FcRs are highly desirable.

### SUMMARY OF THE INVENTION

In a first aspect, the invention provides a fusion polypeptide comprising
a domain derived from an immunoglobulin heavy chain constant region; and
a plurality of hinge regions derived from IgG hinge sequences.

In a second aspect, the invention provides a protein, wherein the protein comprises two fusion polypeptides of the first aspect. Suitably fusion proteins of the invention may comprise more than two fusion polypeptides, to give rise to multimeric or polymeric proteins of the invention, as discussed further below.

In a third aspect, the invention provides a pharmaceutical composition comprising the fusion polypeptide of the first aspect and/or a protein of the second aspect.

In a fourth aspect, the invention provides a fusion polypeptide of the first aspect, a protein of the second aspect, and/or a pharmaceutical composition of the third aspect for use as a medicament.

In a fifth aspect, the invention provides a nucleic acid encoding the fusion polypeptide of the first aspect.

In a sixth aspect, the invention provides a method of producing a fusion polypeptide in accordance with the first aspect of the invention, the method comprising expressing the nucleic acid in accordance with the fifth aspect in a host cell.

### DETAILED DESCRIPTION OF THE INVENTION

The present invention is based upon the inventors' development of novel hinge sequences. Such hinge sequences may be incorporated in the fusion polypeptides of the first aspect of the invention. These hinge sequences may be particularly useful in the context of polymeric proteins, such as polymeric Fc-fusion proteins, as explained in more detail below. Such fusion proteins may be fusion proteins in accordance with the second aspect of the invention.

Multimeric Fc-fusion proteins (generally herein referred to herein by the equivalent term “polymeric proteins”) are highly desirable due to their ability to carry a large number of payload moieties. In the context of treatment, this may allow the provision of a larger number of therapeutic agents at high local densities. In a diagnostic context, the presence of a larger number of payload moieties, such as antigen binding sites, may allow the development of more sensitive assays. Generally, each polypeptide chain can be associated with a separate payload moiety. Accordingly, it will be recognised that hexameric Fc-fusion proteins, are able to carry up to twelve payload moieties per protein hexamer, while dodecamers are able to carry up to twenty four payload moieties.

However, the major disadvantage of the previously described polymeric proteins is associated with their inability to bind FcRs. This significantly reduces the utility of the polymeric Fc-fusion proteins known in the art.

Whilst in some cases the lack of binding to FcRs may be preferred, for example in the context of IVIG treatment, in most cases, binding to these receptors may be highly desirable. Binding to FcRs may increase the Fc-fusion protein's half-life, which prolongs therapeutic and/or vaccine activity, owing to its interaction with the salvage neonatal Fc-receptor, and slower renal clearance of larger molecules. Furthermore, the interaction with FcRs, especially those found on immune cells, may be particularly important for their use in oncological therapies and vaccines.

The novel hinge sequences developed by the inventors', have enabled the production of polymeric Fc-fusion proteins, which, surprisingly, are able to bind FcRs. To the best of the inventors' knowledge, such polymeric Fc-fusion proteins with these binding characteristics have never been reported before.

Furthermore, the hinge sequences may confer a number of additional advantages. For example, the inventors' believe that the hinge sequences may be able to avoid the induction of immunogenic reactions, due to being mainly derived from human hinge sequences.

Additionally, the inventors believe that the length of the hinge sequences is particularly relevant. Hinges which are too long may be susceptible to protease degradation, while hinges which are too short may not create enough separation between the payload moiety and the Ig heavy chain constant region to enable FcR binding. The hinge sequences described herein are of sufficient length to enable FcR binding, while still potentially being resistant to protease degradation.

It will be appreciated that, whilst the hinge sequences developed by the inventors have particular utility in the context of polymeric Fc-fusion proteins, they may also be useful in monomeric Fc-fusion proteins. For example, as explained in more details in the Examples section of this specification, the inventors have shown that the novel hinge sequences increase the binding of monomeric Fc-fusion proteins to FcγRs. This property, may be expected to increase the monomeric Fc-fusion proteins' plasma half-life. The following pages will provide more details of suitable embodiments of the fusion polypeptide, and other aspects of the invention. Except for where the context requires otherwise, embodiments described with reference to one aspect of the invention may also be applied to other aspects of the invention.

### DESCRIPTION OF THE DRAWINGS

FIG. 1A to 1D Schematic representation of the constructs utilised to incorporate four hinge regions into a polypeptide comprising a domain derived from an immunoglobulin heavy chain constant region and IgM tailpiece.

FIG. 2A to 2D Schematic representation of the constructs utilised to incorporate a payload moiety into a polypeptide comprising four hinge regions, a domain derived from an immunoglobulin heavy chain constant region and IgM tailpiece.

FIG. 3A A graph showing the results of a binding assay to determine binding of exemplary polymeric proteins of the invention and comparator proteins to FcγRIIa. B A graph showing the results of a binding assay to determine binding of exemplary polymeric proteins the invention and comparator proteins to FcγRIIIa.

FIG. 4A A graph showing the results of a binding assay to determine binding of exemplary monomeric proteins of the invention and comparator proteins to FcγRIIa. B A graph showing the results of a binding assay to determine binding of exemplary monomeric proteins the invention and comparator proteins to FcγRIIIa.

FIG. 5 illustrates Western blot data confirming the hexameric (FIG. 5A) or monomeric (FIG. 5B) structure of Fc-fusion proteins comprising the hinge sequences.

FIG. 6 graphs illustrating the results of binding assays of the polypeptide of the invention with an adhiron to FcγRI, FcγRIIa, FcγRIIb, FcγRIIIa, and FcγRIIIb receptor.

FIG. 7 graphs illustrating the results of binding assays of the polypeptide of the invention with a Dengue virus antigen to FcγRI, FcγRIIa, FcγRIIb FcγRIIIa, and FcγRIIIb receptor.

FIG. 8 graphs illustrating the results of binding assays of the polypeptide of the invention with a Zika virus antigen to FcγRI, FcγRIIa, FcγRIIb FcγRIIIa, and FcγRIIIb receptor.

FIG. 9 graphs illustrating the results of binding assays of the polypeptide of the invention comprising Dengue EPDIII monomers to mouse gamma receptors, FcγRIIIab and FcγRIV.

FIG. 10 graphs illustrating the results of binding assays of the polypeptide of the invention comprising Zika EPDIII monomers to mouse gamma receptors, FcγRIIIab and FcγRIV.

FIG. 11 graphs illustrating the results of binding assays of the polypeptide of the invention comprising Zika EPDIII monomers or Dengue EPDIII monomers to human DC-SIGN receptors.

A Fusion Polypeptide of the Invention

An example of a fusion polypeptide of the invention is set out in SEQ ID NO: 3. A further exemplary fusion polypeptide of the invention is set out in SEQ ID NO: 4. A fusion polypeptide of the invention may comprise SEQ ID NO: 3 or SEQ ID NO: 4. In a suitable embodiment, a fusion polypeptide of the invention may consist of SEQ ID NO: 3 or SEQ ID NO: 4.

Suitably a fusion polypeptide in accordance with the invention may share at least 70% identity with SEQ ID NO: 3, suitably at least 75%, at least 80%, or at least 85% identity. A fusion polypeptide in accordance with the invention may share at least 90% identity with SEQ ID NO: 3, at least 91%, at least 92%, at least 93%, at least 94%, at least 95%, at least 96%, at least 97%, at least 98%, or at least 99% identity with SEQ ID NO: 3.

Suitably a fusion polypeptide in accordance with the invention may share at least 70% identity with SEQ ID NO: 4, suitably at least 75%, at least 80%, or at least 85% identity. A fusion polypeptide in accordance with the invention may share at least 90% identity with SEQ ID NO: 3, at least 91%, at least 92%, at least 93%, at least 94%, at least 95%, at least 96%, at least 97%, at least 98%, or at least 99% identity with SEQ ID NO: 4.

As described elsewhere in this specification, the fusion polypeptide of the present invention may comprise a payload moiety. The payload moiety may be located at the C-terminus of the domain derived from an immunoglobulin heavy chain constant region. In an embodiment where a payload moiety is present, the payload moiety is separated from the domain by a plurality of hinge regions. Such exemplary fusion polypeptide of the invention which comprises a payload moiety is set out in SEQ ID NO: 5 and SEQ ID NO: 6.

Since the fusion proteins of the invention incorporate fusion polypeptides of the invention, it will be appreciated that (except for where the context requires otherwise), disclosures set out in connection with the fusion polypeptides of the invention are also applicable to fusion proteins of the invention, and vice versa.

A Protein of the Invention

The second aspect of the invention provides a protein comprising two fusion polypeptides of the first aspect. This may be a homodimer of two fusion polypeptides of the invention. However, as discussed further below, in the context of the present disclosure a protein of the invention which comprises two fusion polypeptides will be referred to herein as a “monomeric protein”, or a “monomer” (since these proteins constitute the building blocks for further polymer formation).

Suitably, two fusion polypeptides of the invention may be linked by an inter-disulphide bond to form a monomeric protein of the invention. Suitably the inter-disulphide bond may be formed between a residue corresponding to residue Cys89 and/or Cys248 of SEQ ID NO: 14.

A monomeric protein of the invention may bind one or more additional monomeric proteins, and thus form a polymeric protein of the invention.

A polymeric protein of the invention may be provided in the form of any suitable multimer, including, but not limited to, dimers, trimers, tetramers, pentamers, hexamers, heptamers, octamers, nonomers, decamers, undecamers, and dodecamers. In a suitable embodiment, a polymeric protein of the invention is in the form of a hexamer.

It shall be appreciated that, for present purposes, a “trimer” would be made up of three “monomers” as referred to above—a total of six chimeric polypeptide chains. A “hexamer” would consist of six monomers, and hence a total of twelve chimeric polypeptide chains.

Whether the protein is monomeric or polymeric may depend upon certain adaptations in the amino acid sequence of the fusion polypeptide. Such adaptations may promote or increase polymerisation of the protein, or inhibit polymerisation of the protein. Suitable adaptations are discussed elsewhere in this specification.

For the purposes of the present disclosure references to promoting or increasing polymerisation should, except for where the context requires otherwise, be taken as referring to an increase in the size of polymeric proteins formed. That is to say, for example, an increase in the proportion of dodecamers formed, as compared to the number of pentamers or hexamers. This increase in the size of polymeric proteins formed may optionally occur in combination with an increase in the proportion of the polypeptides being incorporated in polymeric proteins.

By the same token, inhibiting polymerisation refers to a decrease in the proportion of protein present in a polymeric form, or an increase in the proportion of the protein that is present in a monomeric form. This may be assessed with reference to the proportion of polymeric or monomeric protein found in an appropriate control protein. Such an appropriate control protein may comprise a polypeptide corresponding to the polypeptide under investigation, save for the modification in question. Alternatively, a suitable control polypeptide may have the amino acid sequence set out in SEQ ID NO: 14.

A Hinge Region

The fusion polypeptides or proteins of the invention comprises a plurality of hinge regions derived from IgG hinge sequences. The hinge regions are located at the C-terminus of the domain derived from an immunoglobulin heavy chain constant region. It will be appreciated that in an embodiment where the fusion polypeptide comprises a payload moiety, the plurality of hinge regions is located between the payload moiety and the C-terminus of the domain derived from an immunoglobulin heavy chain constant region.

The plurality of hinge regions may increase the distance between the domain derived from an immunoglobulin heavy chain constant region and the payload moiety, if such a payload moiety is present. Increased distance between the domain and the payload moiety may enable the binding of the domain to FcRs (for example FcγIIa and FcγIIIa). The inventors believe that the plurality of hinge regions may provide sufficient space and flexibility between the domain and the payload moiety to allow the attachment of a glycan molecule to a glycosylation site on the fusion polypeptides or proteins of the invention. The presence of a glycan molecule may enable the polypeptides and proteins of the invention to bind FcRs and/or glycan receptors. The inventors also believe that the location of the glycosylation site may influence whether the fusion polypeptides and proteins of the invention bind FcRs, glycan receptors (for example sialic acid receptors such as SIGLEC-1), or both. Suitable glycosylation sites, and the ways in which they may influence the binding of the polypeptides and proteins of the invention, are discussed elsewhere in this specification.

In a suitable embodiment, a hinge region may be derived from an immunoglobulin G selected from the group consisting of IgG1, IgG2, IgG3 and IgG4. A hinge region based upon that of immunoglobulin IgG1 is particularly suitable for incorporation in the fusion polypeptides of the invention. Suitably, a hinge region may be derived from human IgG, such as IgG1. By way of example, the polypeptide of the invention set out in SEQ ID NO: 3, comprises a hinge region derived from human IgG1.

In a suitable embodiment a hinge region may be at least 4, at least 5, at least 6, at least 7, least 8, at least 9, at least 10, at least 11, least 12, at least 13, at least 14, at least 15, least 16, at least 17, at least 18, at least 19, at least 20, or more, amino acids long. Suitably, the hinge region is at least 10 amino acids long.

In a suitable embodiment a hinge region may be 4, 5, 6, 7, 8, 9, 10, 11, 12, 13, 14, 15, 16, 17, 18, 19, 20, or more, amino acids long. Suitably, the hinge region is 10 amino acids long.

In a suitable embodiment, a fusion polypeptide of the invention comprises at least 2, at least 3, at least 4, at least 5, at least 6, at least 7, at least 8, at least 9, at least 10, or more hinge regions. Suitably, the fusion polypeptide of the invention comprises at least 2 hinge regions. Suitably, the fusion polypeptide of the invention comprises at least 3 hinge regions. More suitably, the fusion polypeptide comprises at least 4 hinge regions.

In a suitable embodiment, the fusion polypeptide of the invention comprises 2, 3, 4, 5, 6, 7, 8, 9, 10, or more hinge regions. Suitably, the fusion polypeptide of the invention comprises 2 hinge regions. Suitably, the fusion polypeptide of the invention comprises 3 hinge regions. More suitably, the fusion polypeptide of the invention comprises 4 hinge regions.

In a suitable embodiment the plurality of hinge regions are the same. Merely by way of example, in such an embodiment, each of the plurality of hinge regions comprises or consists of the amino acid sequence set out in SEQ ID NO: 1. Alternatively, each of the plurality of hinge regions comprises or consists of the amino acid sequence set out in SEQ ID NO: 2.

In an alternative embodiment, some or all of the plurality of hinge regions may differ from one another. Merely by way of example, in such an embodiment, some of the plurality of hinge regions may comprise or consist of the amino acid sequence set out in SEQ ID NO:1, while others may comprise or consist of the amino acid sequence set out in SEQ ID NO: 2. In such an embodiment, the hinge region(s) in closer proximity to the domain derived from an immunoglobulin heavy chain constant region may comprise or consist of the amino acid sequence set out in SEQ ID NO: 1. Suitably, the hinge regions further away from the domain derived from an immunoglobulin heavy chain constant region, thus closer to a payload moiety, if present, may comprise or consist of the amino acid sequence set out in SEQ ID NO: 2.

Merely by way of example, in a suitable embodiment one of the hinge regions or two of the hinge regions proximal to the domain derived from an immunoglobulin heavy chain constant region may have the sequence of SEQ ID NO: 1. Similarly, and suitably in the same embodiment, one, two, three or more of the hinge regions proximal to the payload moiety may have the sequence of SEQ ID NO: 2.

As touched upon elsewhere in this specification, the inventors believe that the presence and location of glycosylation sites, which may ultimately determine the presence and location of glycan molecules, may influence whether the fusion polypeptides and proteins of the invention bind FcRs, glycan receptors, or both.

For example, the presence of a glycosylation site within a hinge region in close proximity to the domain derived from an immunoglobulin heavy chain constant region may promote glycan receptor binding, while at the same time preventing FcRs binding.

A suitable glycosylation site in keeping with this embodiment may be located in the hinge region closest to the domain derived from an immunoglobulin heavy chain constant region. Merely by way of example, in a fusion polypeptide of the invention comprising four hinge regions set out by SEQ ID NO:1 or SEQ ID NO: 2, the glycosylation site may be at the amino acid residue corresponding to D037 of SEQ ID NO: 3 or SEQ ID NO: 5.

The presence of a glycosylation site further away from the domain derived from an immunoglobulin heavy chain constant region may allow binding to FcRs, or FcRs and glycan receptors.

A suitable glycosylation site in keeping with this embodiment may be located in one or more hinge regions other than that closest to the domain derived from an immunoglobulin heavy chain constant region. The glycosylation site may be located towards or at the N-terminal end of the hinge region. Merely by way of example, in a fusion polypeptide of the invention comprising four hinge regions set out by SEQ ID NO: 1 or SEQ ID NO: 2, the glycosylation site may be at an amino acid residue corresponding to an amino acid selected from the group consisting of D001, D013 and D025 of SEQ ID NO: 3 or SEQ ID NO: 5. Suitably a glycosylation site may be located at one, more than one, or all of these sites.

In a suitable embodiment, as glycosylation site may be an artificial glycosylation site. Such an artificial glycosylation site may involve a substitution at a residue corresponding to D1 of SEQ ID NO: 1 or 2 (in turn D001, D013, D025 and/or D037 of SEQ ID NO: 3 or SEQ ID NO:5). Suitably, an artificial glycosylation site may be obtained by an aspartic acid to asparagine substitution (for example a DOO1N substitution in SEQ ID NO: 1 or SEQ ID NO: 2, corresponding to D001N, D013N, D025N and/or D037N of SEQ ID NO: 3 or SEQ ID NO:5).

In a protein of the invention (for example a monomer which comprises a first and second fusion protein of the invention) where the hinge regions contain one or more cysteine residues, the hinge regions of the first fusion polypeptide may bind the hinge regions of the second fusion polypeptide. Such binding may be via an inter-disulphide bond. It will be appreciated that when the hinge regions of at least one of the first and/or second fusion polypeptide forming the monomeric protein lacks cysteine residues such binding may be prevented. In a suitable embodiment some or all of the plurality of hinge regions do not comprise one or more cysteine residues. Suitably, some or all of the plurality of hinge regions do not comprise any cysteine residues. Suitably, cysteine residues which otherwise may be present in the corresponding native IgG hinge sequences from which the hinge regions are derived, may be removed by substitution, deletion and/or other modification. Suitably, the cysteine residue(s) may be substituted by an alanine residue.

The inventors believe that the removal of a cysteine residue(s) in one or more hinge regions is advantageous as it may increase the flexibility of the hinge regions and thereby impart greater flexibility between the domain derived from the heavy chain constant region, and the payload moiety, if present. This in turn, may enable the use of larger payload moieties and/or increase the reach of payload moieties to their ligands. Furthermore, the inventors also believe that hinge regions with reduced or no cysteine residues may be more resistant to cleavage by a number of proteases, which cleave peptides near cysteine residues.

It will be appreciated that hinge region flexibility may be particularly desirable in at least one of the hinge regions which are adjacent to the payload moiety. Accordingly, by way of example, in a fusion polypeptide of the invention which comprises four hinge regions, the hinge region closest to the domain derived from an immunoglobulin heavy chain consent region may contain cysteine residues, while the remaining three hinge regions do not. Alternatively, in a fusion polypeptide of the invention which comprises four hinge regions, the two hinge regions closest to the domain derived from an immunoglobulin heavy chain consent region may contain cysteine residues, while the remaining two hinge regions do not.

In a suitable embodiment, a hinge region comprises the human IgG1 hinge sequence DKTHTCPPCP (SEQ ID NO: 1). Alternatively, a hinge region may comprise the sequence DKTHTAPPAP (SEQ ID NO: 2) derived from the human IgG1 hinge sequence. It will be appreciated that in a fusion polypeptide of the invention which comprises a plurality of hinge regions, there may be hinge regions which comprise SEQ ID NO: 1 or SEQ ID NO: 2 within a single fusion polypeptide.

In a suitable embodiment, a hinge region comprises of amino acid sequence -X₁KTHTX₂PPX₂P-.

Suitably, a hinge region comprises of amino acid sequence -X₁KTHTX₂PPX₂P-, wherein is aspartic acid.

Suitably, a hinge region comprises of amino acid sequence -X₁KTHTX₂PPX₂P-, wherein X₁ is asparagine.

Suitably, a hinge region comprises of amino acid sequence -X₁KTHTX₂PPX₂P-, wherein X₂ is cysteine.

Suitably, a hinge region comprises of amino acid sequence -X₁KTHTX₂PPX₂P-, wherein X₂ is an amino acid other than cysteine. Suitably, the amino acid other than cysteine is alanine. Suitably, a hinge region comprises of amino acid sequence -X₁KTHTX₂PPX₂P-, wherein X₁ is aspartic acid and X₂ is cysteine.

Suitably, a hinge region comprises of amino acid sequence -X₁KTHTX₂PPX₂P-, wherein X₁ aspartic acid, and X₂ is an amino acid other than cysteine. Suitably, the amino acid other than cysteine is alanine.

Suitably, a hinge region comprises of amino acid sequence -X₁KTHTX₂PPX₂P-, wherein X₁ is asparagine and X₂ is cysteine.

Suitably, a hinge region comprises of amino acid sequence -X₁KTHTX₂PPX₂P-, wherein X₁ asparagine, and X₂ is an amino acid other than cysteine. Suitably, the amino acid other than cysteine is alanine.

In a suitable embodiment of the invention, a hinge region consists of amino acid sequence -KTHTX₂PPX₂P-.

Suitably, a hinge region consists of amino acid sequence -X₁KTHTX₂PPX₂P-, wherein X₁ is aspartic acid.

Suitably, a hinge region consists of amino acid sequence -X₁KTHTX₂PPX₂P-, wherein X₁ is asparagine.

Suitably, a hinge region consists of amino acid sequence -X₁KTHTX₂PPX₂P-, wherein X₂ is cysteine.

Suitably, a hinge region consists of amino acid sequence -X₁KTHTX₂PPX₂P-, wherein X₂ is an amino acid other than cysteine. Suitably, the amino acid other than cysteine is alanine.

Suitably, a hinge region consists of amino acid sequence -X₁KTHTX₂PPX₂P-, wherein X₁ is aspartic acid and X₂ is cysteine.

Suitably, a hinge region consists of amino acid sequence -X₁KTHTX₂PPX₂P-, wherein X₁ aspartic acid, and X₂ is an amino acid other than cysteine. Suitably, the amino acid other than cysteine is alanine.

Suitably, a hinge region consists of amino acid sequence -X₁KTHTX₂PPX₂P-, wherein X₁ is asparagine and X₂ is cysteine.

Suitably, a hinge region consists of amino acid sequence -X₁KTHTX₂PPX₂P-, wherein X₁ asparagine, and X₂ is an amino acid other than cysteine. Suitably, the amino acid other than cysteine is alanine.

In an embodiment, where a hinge region comprises or consists of amino acid -X₁KTHTX₂PPX₂P-, wherein X₁ is asparagine, the hinge region may be glycosylated at this position. Such a glycosylation may inhibit polymerisation of a protein in accordance with this aspect of the invention.

Suitably, the hinge region may comprise SEQ ID NO: 1 or SEQ ID NO: 2. Suitably, the hinge region may consist of SEQ ID NO: 1 or SEQ ID NO: 2.

In a suitable embodiment one or more, or all of the plurality of hinge regions may be separated from each other by a short amino acid sequence.

Suitably, the short amino acid sequence may be up to 10, up to 9, up to 8, up to 7, up to 6, up to 5, up to 4, up to 3, up to 2, or 1 amino acid residues in length. Suitably, one or more, or all, of the hinge regions may be separated from one another by short amino acid sequences of 2 amino acid residues.

Suitably, the short amino acid sequence may be selected from the group consisting of RS, SR, and VD. It will be appreciated that when more than two hinge regions are present within the hinge area, the hinge regions may be separated by different short amino acid sequences. For example, in an embodiment where the fusion polypeptide comprises four hinge regions, the first and second hinge regions may be separated by the amino acid sequence SR, the second and third hinge regions may be separated by the amino acid sequence VD, and the third and fourth hinge regions may be separated by an amino acid RS.

In a suitable embodiment the short amino acid sequence may comprise a restriction site. The presence of a restriction site may enable or simplify the addition and/or removal of one or more hinge regions.

Furthermore, the inventors believe that the presence of a short amino acid sequence between the hinge regions may confer a number of other advantages. For example, the short amino acid sequence may allow for the insertion of a glycan sequon without interfering with the structure of the hinge region(s). Suitably, the glycan sequon may be according to SEQ ID NO: X (N-X₁-X₂). Suitably, X₁ may be any amino acid. Suitably, X₂ may be a threonine or a serine.

Additionally or alternatively, the short amino acid sequence may render the hinge region(s) resistant to cleavage by a number of proteases. Suitably, in such an embodiment, the short amino acid sequences may disrupt restriction sites that would otherwise be present.

Surprisingly, the inventors have found that the short amino acid sequence may confer the above mentioned advantages without adversely affecting the ability of the protein of the invention to bind a Fc receptor, even when said protein comprises a payload moiety.

A Domain Derived from an Immunoglobulin Heavy Chain Constant Region

The fusion polypeptide of the invention incorporates a domain derived from an immunoglobulin heavy chain constant region. The immunoglobulin heavy chain constant region may be from any suitable source. Suitably the domain may be derived from the heavy chain constant region of a mammalian immunoglobulin, for example a human or murine immunoglobulin. In a suitable embodiment, the domain derived from an immunoglobulin heavy chain constant region is derived from an Ig selected from the group consisting of IgG, IgA, IgM and IgE. Suitably the Ig is IgG.

A suitable IgG may be selected from the group consisting of: IgG1; IgG2; IgG3; and IgG4. In a suitable embodiment the IgG is IgG1.

In a suitable embodiment the fusion polypeptide of the invention comprises a plurality of domains derived from an immunoglobulin heavy chain constant region(s). Suitably, the polypeptide may comprise, 2, 3, 4 or more domains derived from an immunoglobulin heavy chain constant region. Suitably, the fusion polypeptide may comprise 2 domains derived from an immunoglobulin heavy chain constant region. More suitably the 2 domains derived from an immunoglobulin heavy chain constant region are derived from CH2 and CH3 domains.

In an embodiment where the fusion polypeptide of the invention comprises a plurality of domains derived from an immunoglobulin heavy chain constant regions, the domains may all be the same as one another. Alternatively, some or all of the plurality of immunoglobulin heavy chain constant domains are different.

It will be appreciated that as long as they meet the requirement of forming an Fc receptor binding portion, the immunoglobulin heavy chain constant region utilised in fusion polypeptide of the invention may include an alteration in its sequence as compared to the native sequence from which it is derived from. Merely by way of example, a suitable fusion polypeptide of the invention may utilise an immunoglobulin (for example IgG) derived sequences that share at least 60%, at least 70%, at least 75%, at least 80%, at least 85%, at least 90%, at least 95%, at least 96%, at least 97%, at least 98%, or at least 99% sequence identity with the relevant native immunoglobulin (for example IgG) sequence from which it is derived.

A Payload Moiety

In a suitable embodiment, the fusion polypeptide of the present invention may comprise a payload moiety. Examples of suitable payload moieties are described below.

The payload moiety may be located at the C-terminus of the domain derived from an immunoglobulin heavy chain constant region and separated from the domain by a plurality of hinge regions. In other words, the plurality of hinge regions are located between the domain derived from an immunoglobulin heavy chain constant region and the payload moiety.

The payload moiety may be naturally occurring or synthetic. A naturally occurring payload moiety is one that can be found in nature. A synthetic payload moiety is one that does not exist in nature (for example one that is manmade).

In a suitable embodiment, a naturally occurring payload moiety is a proteinaceous molecule, or a non-proteinaceous molecule.

In a suitable embodiment, a naturally occurring or synthetic payload moiety may be selected from the group consisting of a chemical compound, a proteinaceous molecule, a nucleic acid molecule, a lipid, and a carbohydrate. It will be appreciated that a synthetic proteinaceous molecule or a synthetic nucleic acid molecule may have a sequence which is derived from a naturally occurring sequence.

A proteinaceous molecule may be selected from the group consisting of a protein, a peptide and a peptidomimetic. By way of example, a proteinaceous molecule may be an antigen, a polypeptide scaffold, a pathogen-associated molecular pattern (PAMP), a ligand, a receptor, a cytokine or a chemokine. Accordingly, a polypeptide according may comprise a payload moiety selected from the group consisting of an antigen, a polypeptide scaffold, a pathogen-associated molecular pattern (PAMP), a ligand, a receptor, a cytokine and a chemokine. It will be appreciated that a fusion protein of the invention comprising an antigen as the payload moiety may be particularly useful in the context of a vaccine.

The antigen may be derived from any suitable pathogenic or non-pathogenic organism. More suitably, the antigen may be derived from a pathogenic organism. More suitably, the pathogenic organism is a virus or bacterium.

Merely by way of example, the antigen may be derived from a virus selected from the group consisting of: Zika virus, Dengue virus, Adeno-associated virus, Chikungunya virus, Cosavirus A, Cowpox virus, Ebolavirus, Echovirus, Encephalomyocarditis virus, Epstein-Barr virus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Influenza A virus, Influenza B virus, Influenza C virus, MERS coronavirus, Measles virus, Mumps virus, Poliovirus, Rabies virus, Rotavirus A, Rotavirus B, Rotavirus C, Rubella virus, Varicella-zoster virus, West Nile virus and Yellow fever virus. Other suitable antigens derived from a virus will be known to those skilled in the art.

Suitably, an antigen derived from a virus may be based on the EPIII domain. For example, an antigen derived from the Zika virus EPIII domain may comprise or consist of SEQ ID NO: 17, whereas an antigen derived from the Dengue virus EPIII domain may comprise or consist of SEQ ID NO: 18.

Merely by way of example, the antigen may be derived from a bacteria selected from the group consisting of Acinetobacter baumannii, Actinomyces israelii, Bacillus anthracis, Bacillus cereus, Bacteroides fragilis, Bartonella henselae, Bartonella quintana, Bordetella pertussis, Borrelia burgdorferi, Borrelia garinii Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Chlamydophila pneumoniae, Clostridium botulinum, Clostridium difficile, Clostridium perfringens, Clostridium tetani, Corynebacterium diphtheriae, Coxiella burnetiid, Enterococcus faecalis, Enterococcus faecium, Escherichia coli, Ehrlichia canis, Ehrlichia chaffeensis, Francisella tularensis, Haemophilus influenzae, Helicobacter pylori, Klebsiella pneumoniae, Legionella pneumophila, Leptospira interrogans, Leptospira santarosai, Leptospira weilii, Leptospira noguchii, Listeria monocytogenes, Moraxella catarrhalis, Mycobacterium leprae, Mycobacterium tuberculosis, Mycobacterium ulcerans, Mycoplasma pneumoniae, Neisseria gonorrhoeae, Neisseria meningitidis, Nocardia asteroids, Pseudomonas aeruginosa, Rickettsia rickettsia, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholera, Yersinia pestis, Yersinia enterocolitica, and Yersinia pseudotuberculosis. Other suitable antigens derived from a bacterium will be known to those skilled in the art.

In a suitable embodiment, the polypeptide scaffold is an adhiron. Suitably, the adhiron may be F1 adhiron. Suitably the F1 adhiron may comprise or consist of SEQ ID NO: 19.

A non-proteinaceous molecule may be selected, for example, from the group consisting of a chemical compound, a nucleic acid molecule, a lipid and a carbohydrate. Other non-proteinaceous molecules will be known to the skilled person.

In a suitable embodiment, a chemical compound may be a small molecule. By way of example and not limitation, suitable small molecules may be selected from the group consisting of mertansine, monomethylauristatin E, doxorubicin and N-acetyl-γ calicheamicin.

In a suitable embodiment, the payload moiety may be fused to the fusion polypeptide or protein of the invention. Alternatively, the payload moiety may be conjugated with the polypeptide or protein of the invention.

In an embodiment where the payload moiety is fused to the fusion polypeptide or protein of the invention, the amino acid sequence encoding the payload moiety is located within the same reading frame as the amino acid sequence of the polypeptide of the invention. The fused payload moiety may thus be expressed as part of the same gene product as the fusion polypeptide. As such, it will be appreciated that a payload moiety that is fused to the fusion polypeptide of the invention is a proteinaceous molecule. Such a fused proteinaceous molecule may be naturally occurring or synthetic.

The term “conjugated” as used herein refers to an interaction between a fusion polypeptide or protein of the invention and a non-proteinaceous molecule by means of covalent bonding, or by means of weak interactions.

In a suitable embodiment, the payload moiety may be selected from the group consisting of: a therapeutic agent; a diagnostic agent; and a research agent. Each of these may be naturally occurring or synthetic.

In a suitable embodiment, a therapeutic agent is a molecule which has a therapeutic effect. Such a therapeutic effect may include amelioration of a symptom and/or disease, delay of onset of a symptom and/or disease, and/or prevention of onset of a symptom and/or disease. The therapeutic effect of a therapeutic agent may be in addition to, or independent of, the therapeutic effect of the polypeptide or protein of the invention.

A therapeutic agent may be selected from the group consisting of a drug, a carbohydrate, a nucleic acid and a proteinaceous molecule.

The term “drug” as used herein refers to a chemical compound with therapeutic activity, for example a small molecule, which may be conjugated to a fusion polypeptide or protein of the invention. Merely by way of example, a suitable drug therapeutic molecule may be one, such as monomethyl auristatin E, which may be useful in the treatment of cancer. Suitably, the drug, such as monomethyl auristatin E, may be further conjugated to an antibody. Accordingly, a polypeptide or protein of the invention may be conjugated to an anti-cancer drug, such as monomethyl auristatin E.

Merely by way of example, a suitable proteinaceous molecule may be a protein, such as a cytokine receptor. Cytokine receptors may be useful for inhibiting disease-causing cytokines, by for example, binding such disease-causing cytokines, and thereby preventing them from pathogenically binding to cells.

In another example, the proteinaceous molecule is a protein which is an immune modulator. A polypeptide or protein of the invention fused or conjugated to an immune modulator which upregulates components of the immune system may be useful as a vaccine. By way of example an immune modulator which may be useful as a vaccine may be a pathogen-associated molecular pattern (PAMP) molecule or an antigen.

A polypeptide or protein of the invention fused or conjugated to an immune modulator which down regulates the components of the immune system may be useful as a medicament for autoimmune diseases, for example rheumatoid arthritis.

An example of such an immune modulator which down regulates the components of the immune system is erythropoietin. Accordingly, it will be appreciated that in a suitable embodiment erythropoietin may be conjugated or fused to a polypeptide or protein of the invention. Such a conjugated protein may be used in the prevention or treatment of an autoimmune disease.

A suitable carbohydrate to be conjugated to the fusion polypeptide or protein of the invention may be, for example, hyaluronic acid.

A suitable nucleic acid to be conjugated to the fusion polypeptide of the invention may be, for example, unmethylated CpG oligodeoxynucleotide. Fusion polypeptides or proteins of the invention conjugated in this manner are suitable for medical use as immunostimulants.

In a suitable embodiment, a diagnostic agent is a molecule or compound which may detect the presence of a target molecule within the subject and/or a test sample. The presence of a target molecule may be indicative of a disease. Suitably, the diagnostic agent may be for use in in vivo and/or in vitro diagnosis. More suitably it may be for use in in vitro diagnosis. Suitably it may be not for use in in vivo diagnosis.

A Tailpiece

The fusion polypeptides or proteins of the invention may comprise a tailpiece. The term “tailpiece” as used herein will be familiar to those skilled in the art. For the avoidance of doubt, it refers to an amino acid sequence located to the N-terminus of the domain derived from an Ig heavy chain constant region.

In a suitable embodiment, a tailpiece may be based upon the tailpiece of an immunoglobulin selected from the group consisting of: IgM, IgA, and IgE. Suitably the tailpiece is based upon the tailpiece of immunoglobulin IgA. More suitably, the tailpiece is based upon the tailpiece of immunoglobulin IgM.

The tailpiece may be derived from the same species as is the domain derived from an immunoglobulin heavy chain constant region. Alternatively, the tailpiece and domain derived from an immunoglobulin heavy chain constant region may be derived from different species. In a suitable embodiment, the tailpiece is based upon a tailpiece of a human immunoglobulin (for example human IgM or human IgA).

Tailpieces suitable for incorporation in the fusion polypeptides or proteins of the invention may share at least 55% identity with a native immunoglobulin tailpiece, such as the IgM tailpiece. Indeed, a suitable tailpiece may share at least 55%, at least 65%, at least 75%, at least 80%, at least 85%, at least 90%, at least 95% or more identity with the sequence of a corresponding portion of a native immunoglobulin tailpiece.

A Spacer

The fusion polypeptides or proteins of the invention may comprise a spacer. Suitably the spacer may be located between domain derived from the Ig heavy chain constant region and the tailpiece.

A suitable spacer may be at least one, at least two, at least three, at least four, at least five, at least six, at least seven, at least eight, at least nine, at least ten, or more amino acid residues long. More suitably, the spacer may be four amino acid residues long.

Suitably the spacer may comprise or consist of the sequence LVLG (SEQ ID NO: 13).

An Adaptation of the Fusion Polypeptide of the Invention

As touched upon elsewhere in this specification, the fusion polypeptide of the invention may comprise an adaptation which promotes or increases the ability of proteins of the invention comprising the polypeptide to polymerise. Alternatively, the fusion polypeptides of the invention may comprise an adaptation which inhibits the ability of proteins comprising the polypeptide to polymerise.

Suitably, an adaptation may be present in one or more of the plurality of hinge regions, and/or in a domain derived from an Ig heavy chain constant region, and/or in the tailpiece, and/or in the spacer. Examples of suitable adaptations are provided below.

In a suitable embodiment the hinge region may comprise an amino acid adaptation.

Suitably, such an amino acid adaptation of the hinge region may inhibit the polymerisation of the protein of the invention.

Alternatively, such an amino acid adaptation of the hinge region may promote the polymerisation of the protein of the invention.

By way of example, an adaptation of the hinge region which inhibits polymerisation of the proteins of the invention may correspond to adaptation D0037N of SEQ ID NO: 3 or SEQ ID NO: 5.

By way of example, an adaptation of the hinge region which promotes the polymerisation of the proteins of the invention may involve the addition of a cysteine residue to one or more hinge regions, for example the hinge regions of SEQ ID NO: 1 or SEQ ID NO: 2. Additionally, or alternatively, an adaptation of the hinge region which promotes the polymerisation of the proteins of the invention may involve the addition of a motif that promotes polymerisation (for example a motif from complement component 4 binding protein—C4BP) to one or more hinge regions.

In a suitable embodiment, a domain derived form an Ig heavy chain constant region may comprise an amino acid adaptation.

Suitably, such an amino acid adaptation of the domain derived form an immunoglobulin heavy chain constant region may inhibit the polymerisation of the proteins of the invention.

Alternatively, such an amino acid adaptation of the domain derived form an immunoglobulin heavy chain constant region may promote the polymerisation of the proteins of the invention. By way of example, an adaptation of the domain derived form an immunoglobulin heavy chain constant region which inhibits polymerisation of the proteins of the invention may be an adaptation of the residue corresponding to C125 of SEQ ID NO: 5 (which in turn corresponds to C89 of SEQ ID NO: 4). The adaptation may be a substitution. Suitably the cysteine residue may be substituted by an alanine residue.

By way of example, an adaptation of the domain derived from an immunoglobulin heavy chain constant region which promotes the polymerisation of the proteins of the invention may correspond to the lysine to cysteine adaptation K89C of SEQ ID NO: 14.

In a suitable embodiment the tailpiece may comprise an amino acid adaptation.

Suitably, such an amino acid adaptation may inhibit the polymerisation of the proteins of the invention.

Alternatively, such an amino acid adaptation may promote the polymerisation of the proteins of the invention.

By way of example, an adaptation of the tailpiece which inhibits polymerisation of the proteins of the invention may be an adaptation of the residue corresponding to C248 of SEQ ID NO: 14. Alternatively, or additionally, the adaptation of the tailpiece may be an adaptation of the residue corresponding to C89 of SEQ ID NO: 14. A suitable adaptation of the residue in question may be a substitution. In the case of the residue corresponding to C248, a suitable substitution may be substitution by an alanine residue. In the case of the residue corresponding to C89, a suitable substitution may be substitution by a leucine residue.

By way of example, an adaptation of the tailpiece which promotes the polymerisation of the proteins of the invention may be an adaptation of the residue corresponding to N236A of SEQ ID NO: 14. Alternatively or additionally, such an adaptation may be an adaptation of the residue corresponding to C248A of SEQ ID NO: 14. A suitable adaptation of either of these residues may be a substitution. Suitably either or both of these residues may be substituted by an alanine residue.

In a suitable embodiment the spacer may comprise an amino acid adaptation.

Suitably, such an amino acid adaptation may inhibit the polymerisation of the proteins of the invention.

Alternatively, such an amino acid adaptation may promote the polymerisation of the proteins of the invention.

The fusion polypeptide may comprise other suitable adaptations. Examples of such adaptations are provided in PCT/GB2017/051212 and PCT/GB2015/052098, the disclosures of which, insofar as they relate to adaptation of polypeptide sequences, are incorporated herein by reference.

A Pharmaceutical Composition

Also provided by the present invention is a pharmaceutical composition comprising a protein of the invention. In embodiments, the composition is a composition comprising the protein of the invention and a pharmaceutically acceptable diluent, carrier or excipient. Such compositions may further routinely contain pharmaceutically acceptable concentrations of salt, buffering agents, preservatives, compatible carriers, supplementary immune potentiating agents such as adjuvants and cytokines and optionally other therapeutic agents.

The compositions may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The protein of the invention may be presented as solids in finely divided solid form, for example they may be micronised. Powders or finely divided solids may be encapsulated.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The protein of the invention may be for administration to the subject by any suitable route by which a therapeutically effective amount of the protein of the invention may be provided.

In one embodiment, the protein of the invention is for oral administration. Suitable oral administration forms that may be used in such embodiments include solid dosage forms. Solid dosage forms for oral administration include capsules, tablets (also called pills), powders and granules. In such solid dosage forms, the protein of the invention typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules and tablets, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, oral formulations may contain a dissolution aid. The dissolution aid is not limited as to its identity so long as it is pharmaceutically acceptable. Examples include nonionic surface agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface agents, such as betaines and aminocarboxylic acid salts. Pharmaceutical compositions of the invention, comprising the protein of the invention, may also be in microencapsulated form, if appropriate, with one or more of the above-mentioned excipients.

In one embodiment, the protein of the invention is for administration in liquid dosage form. Liquid dosage forms for oral administration include pharmaceutically acceptable emulsions, solutions, suspensions, syrups and elixirs. In addition to the protein of the invention, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavouring and perfuming agents. Suspensions, in addition to the protein of the invention may contain suspending agents such as ethoxylated isostearyl alcohols, polyoxyethylene sorbitol and sorbitan esters, microcrystalline cellulose, aluminium metahydroxide, bentonite, agar-agar, and tragacanth and mixtures thereof.

In one embodiment the protein of the invention may be for administration to the subject by intravenous route. In such an embodiment, a sterile pharmaceutical composition may be especially desirable.

A sterile pharmaceutical composition may be created, for example, by filtration through sterile filtration membranes, prior to or following lyophilisation and reconstitution of the protein. The protein of the invention may be stored in lyophilised form or in solution.

A pharmaceutical composition comprising the protein of the invention may be placed into a container having a sterile access port, for example, an intravenous solution bag or vial having an adapter that allows retrieval of the formulation, such as a stopper pierce-able by a hypodermic injection needle.

A sterile pharmaceutical composition comprising the protein of the invention suitable for intravenous delivery may be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

In a suitable embodiment the pharmaceutical composition comprising the protein of the invention may be for the sustained release of the protein. Such a pharmaceutical composition may comprise semipermeable matrices of solid hydrophobic polymers containing the protein, which matrices are in the form of shaped articles, films or microcapsules. Examples of sustained-release matrices include polyesters, hydrogels, copolymers of L-glutamic acid and gamma ethyl-L-glutamate, non-degradable ethylene-vinyl acetate, degradable lactic acid-glycolic acid copolymers such as the LUPRON Depot™ (injectable microspheres composed of lactic acid-glycolic acid copolymer and leuprolide acetate), and poly-D-(-)-3-hydroxybutyric acid.

In another embodiment, pharmaceutical compositions for sustained release of the protein of the invention, may comprise crystals of the protein suspended in suitable formulations capable of maintaining crystals in suspension. Such pharmaceutical compositions, when injected intravenously, subcutaneously or intraperitoneally may produce a sustained release effect.

A Nucleic Acid of the Invention

The fifth aspect of the invention provides nucleic acids that encode the fusion polypeptides of the invention.

The nucleic acid of the invention may be a DNA molecule encoding fusion polypeptide of the invention. Alternatively, the nucleic acid of the invention may be an RNA molecule, encoding a fusion polypeptide of the invention.

Suitably, a nucleic acid of the invention may comprise SEQ ID NO: 15, which encodes a polypeptide of SEQ ID NO: 3.

In a suitable embodiment the nucleic acid of the invention may share at least 70% identity with SEQ ID NO: 15, at least 75% identity with SEQ ID NO: 15, at least 80% identity with SEQ ID NO: 15, at least 85% identity with SEQ ID NO: 15, at least 90% identity with SEQ ID NO: 15, at least at least 95% identity with SEQ ID NO: 15, at least 96% identity with SEQ ID NO: 15, at least 97% identity with SEQ ID NO: 15, at least 98% identity with SEQ ID NO: 15, or at least 99% identity with SEQ ID NO: 15.

Suitably, a nucleic acid of the invention may comprise SEQ ID NO: 16, which encodes a polypeptide of SEQ ID NO: 5.

In a suitable embodiment the nucleic acid of the invention may share at least 70% identity with SEQ ID NO: 16, at least 75% identity with SEQ ID NO: 16, at least 80% identity with SEQ ID NO: 16, at least 85% identity with SEQ ID NO: 16, at least 90% identity with SEQ ID NO: 16, at least at least 95% identity with SEQ ID NO: 16, at least 96% identity with SEQ ID NO: 16, at least 97% identity with SEQ ID NO: 16, at least 98% identity with SEQ ID NO: 16, or at least 99% identity with SEQ ID NO: 16.

It will be appreciated the nucleic acids of the invention may be incorporated in larger nucleic acid sequences, which will comprise regions that do not encode the fusion polypeptides of the invention. Merely by way of example, a nucleic acid of the invention may be incorporated in an expression plasmid, such as pFUSE-hIgG1-Fc-TP-LH309/310CL or pFUSE-hIgG1-Fc-TP-L310H.

A Method of Production

The sixth aspect of the invention provides a method of producing a fusion polypeptide in accordance with the first aspect of the invention. These methods comprise expressing a nucleic acid in accordance with the fifth aspect of the invention in a host cell.

In a suitable embodiment, the host cell may be a eukaryotic host cell. In particular, a suitable eukaryotic expression host may be selected from the group consisting of yeasts (for example Pichia pastoris and Saccharomyces cerevisiae) and mammalian cell systems. Suitable mammalian cell systems may be selected from the group consisting of: HEK-293 cells, CHO-K1 cells, mouse-derived NSO cells and BHK cells. Other suitable mammalian cell systems will be known to the skilled person. It will be appreciated that suitable host cells will comprise a means for attaching glycans to the expressed proteins.

The invention will now be further described with reference to the following Examples.

### EXAMPLES

1. Methods

Method of Generating the Hinge Cloning Constructs

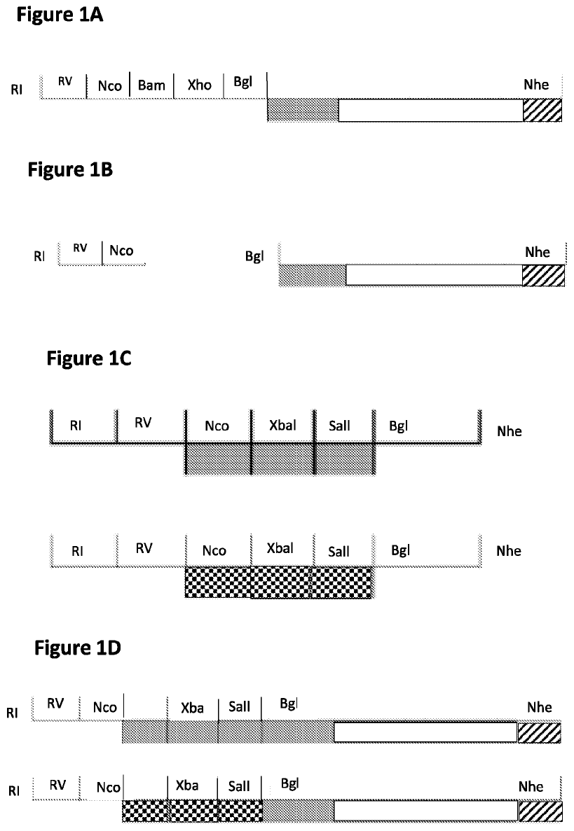

A trimer hinge-repeat was synthesised commercially and cloned into a parking vector that contained all relevant restriction enzyme sites. The trimer repeat was then sub-cloned as Ncol-BgIII fragment in front of the existing hinge on the IgG-Fc hexamer or monomer expression plasmids as published previously (FIG. 1A-D).

The trimer hinge repeats were synthesised with cysteines (3H) or with cysteines replaced by alanines (3HF).

1.1 Method of Fusing a Payload Moiety

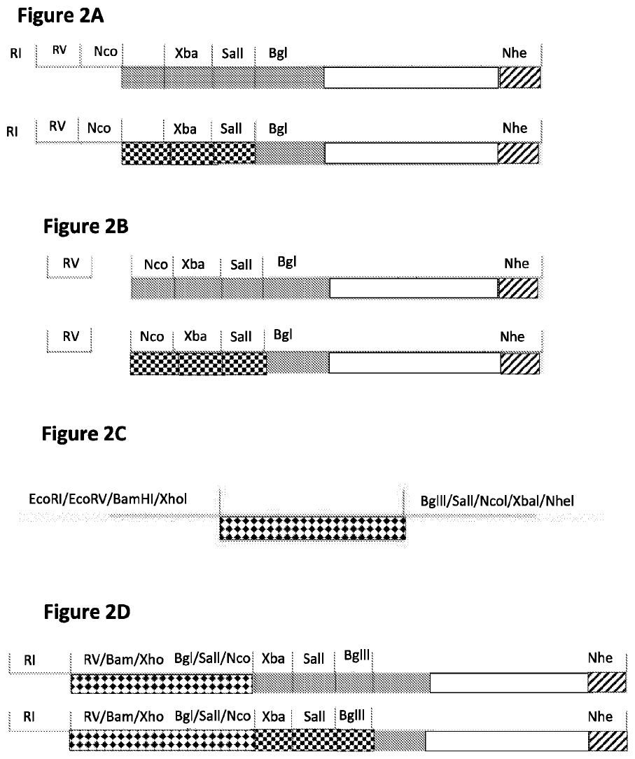

The DNA encoding the fusion partner was synthesised commercially and sub-cloned into a parking vector pFMCS prior to sub-cloning as an EcoRV-Ncol fragment into expression cassettes containing the human IgG1-Fc sequence and the 4H or 4HF repeats (FIG. 2A-D). The resulting plasmids were transfected into CHO-K1 mammalian cells and selected with Zeocin. Clones expressing Fc-proteins were selected and expanded in culture medium. Proteins were harvested by protein-G affinity chromatography.

1.2 Method of Measuring Binding to Fc-Gamma Receptors

Binding of monomeric and polymeric Fc-fusion proteins to FcγRI, FcγRIIa, FcγRIIb, FcγRIIIa, and FcγRIIIb, as well as binding to DC-SIGN was determined by ELISA as described in Blundell et al, JBC 2017.

2. Results

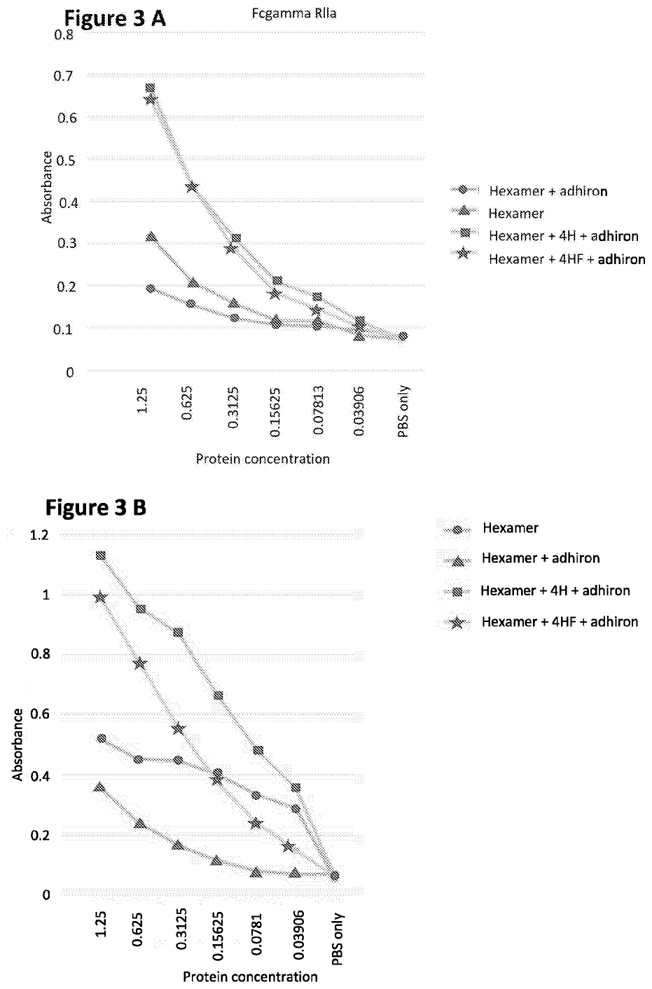

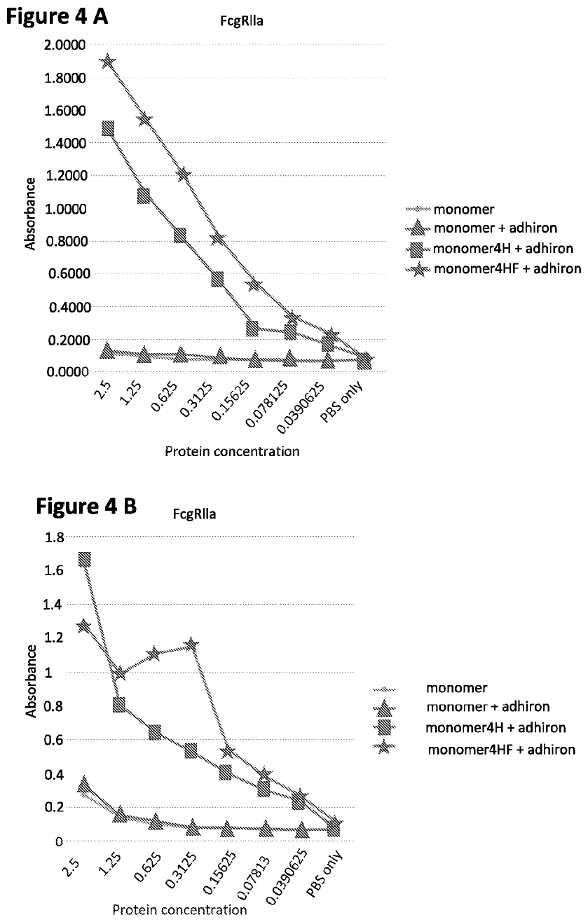

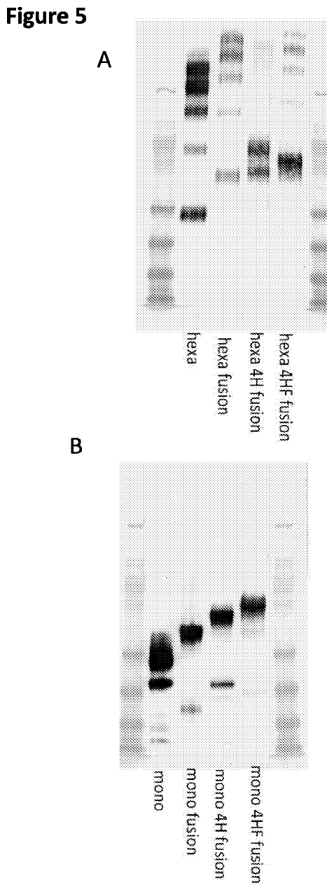

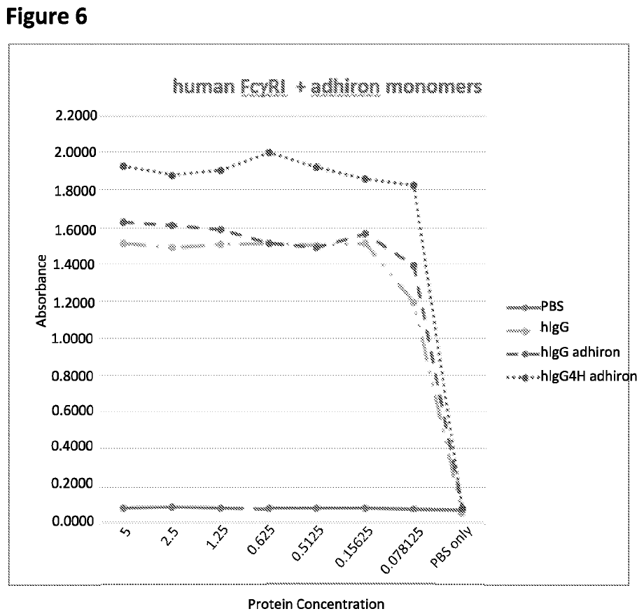

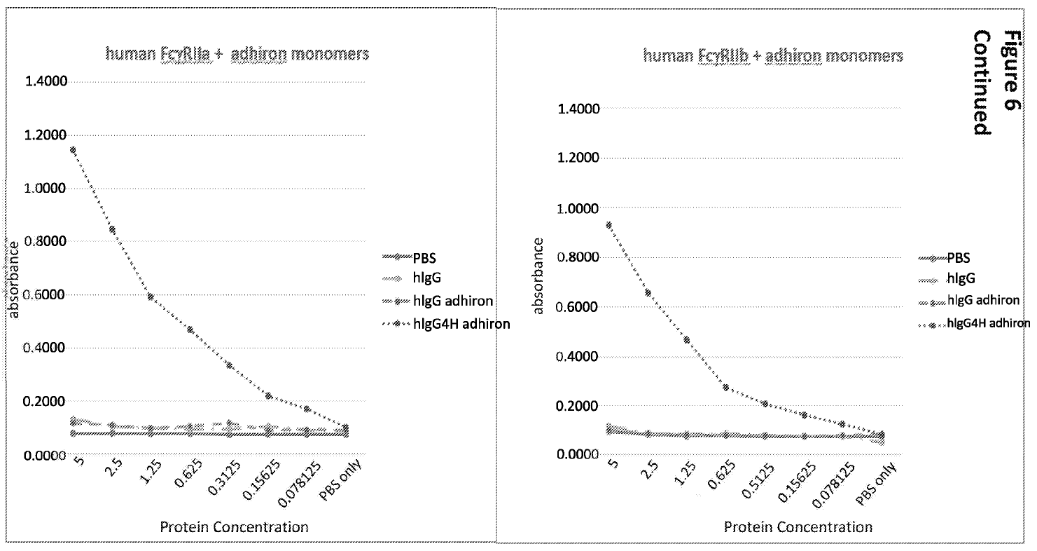

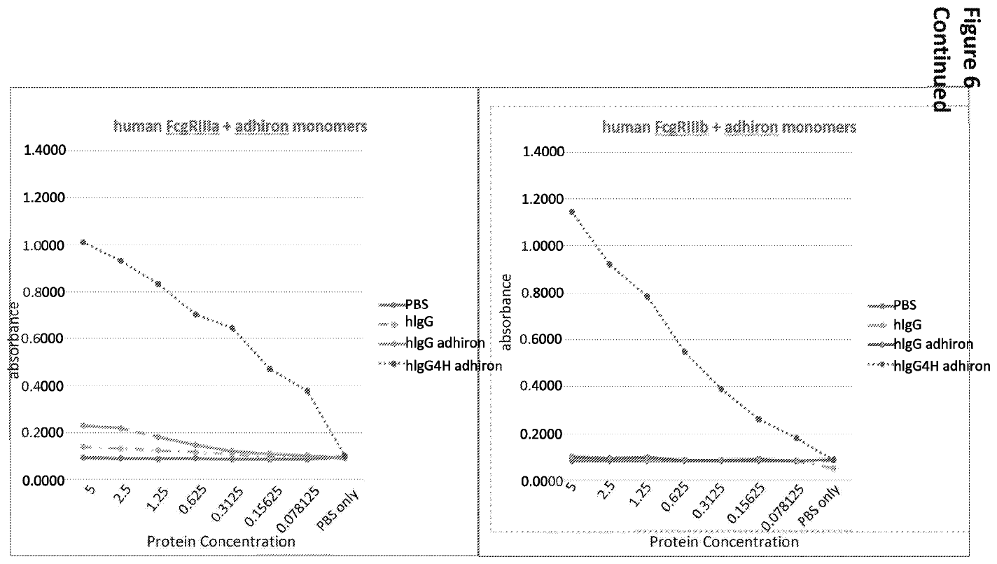

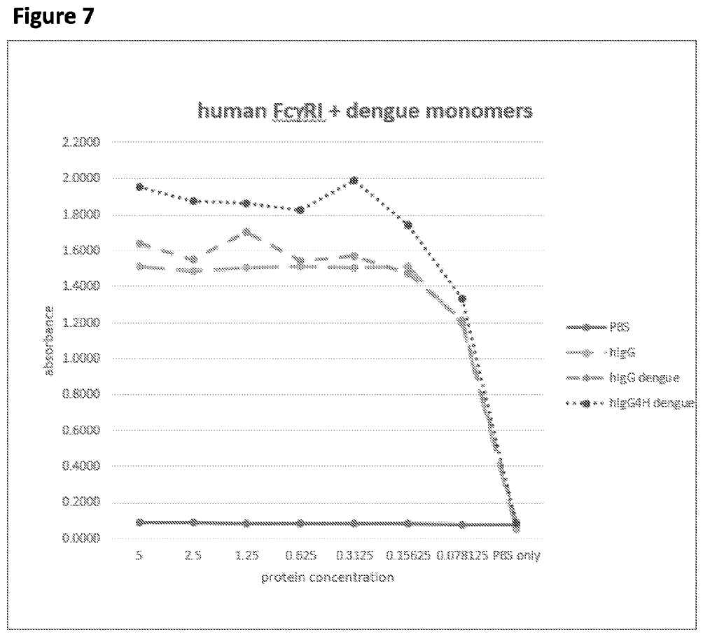

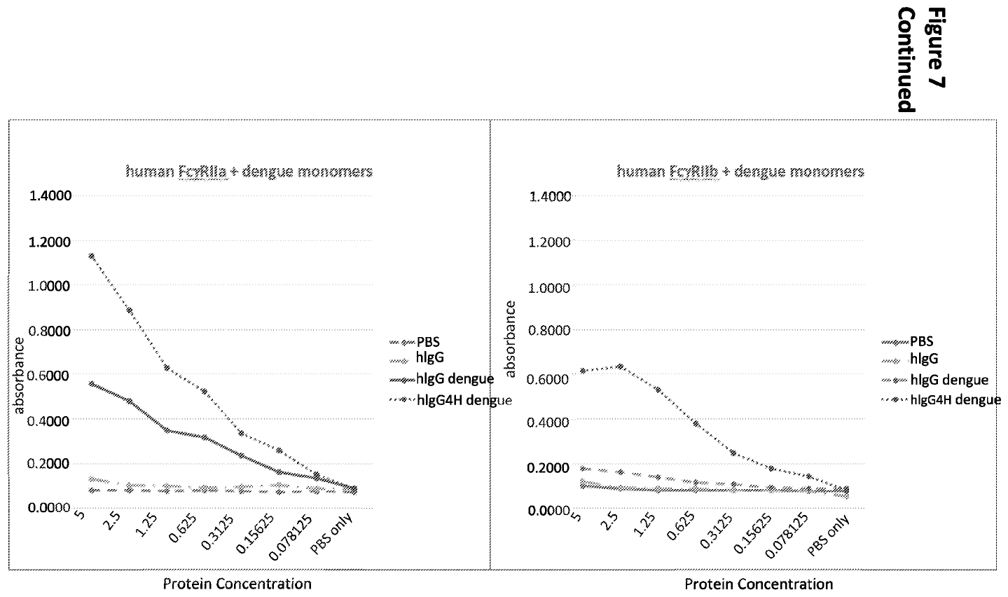

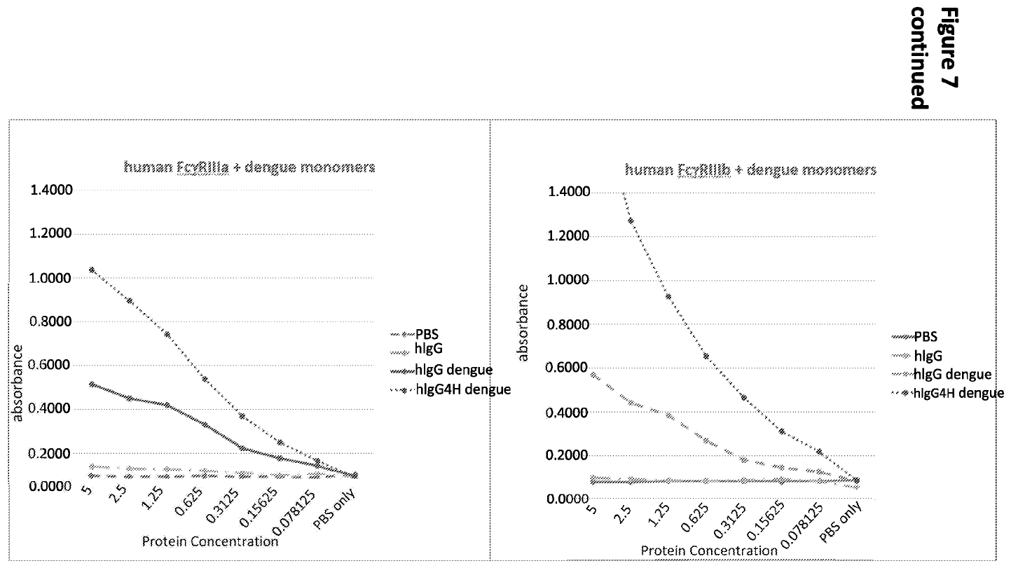

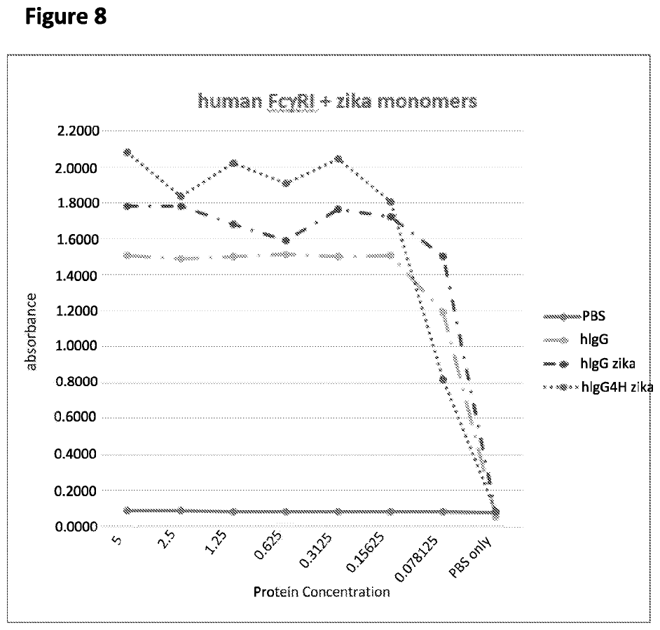

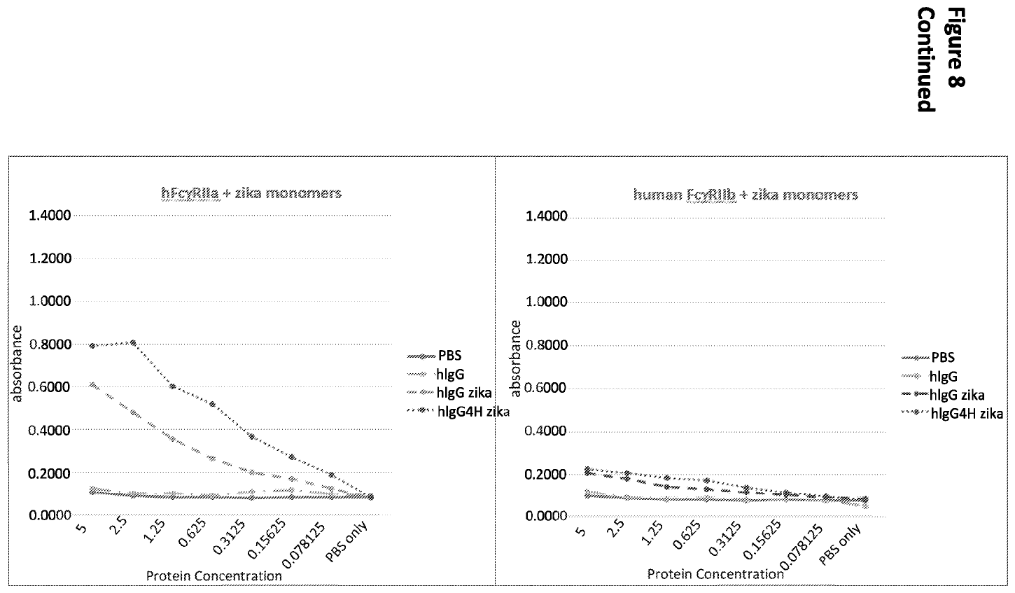

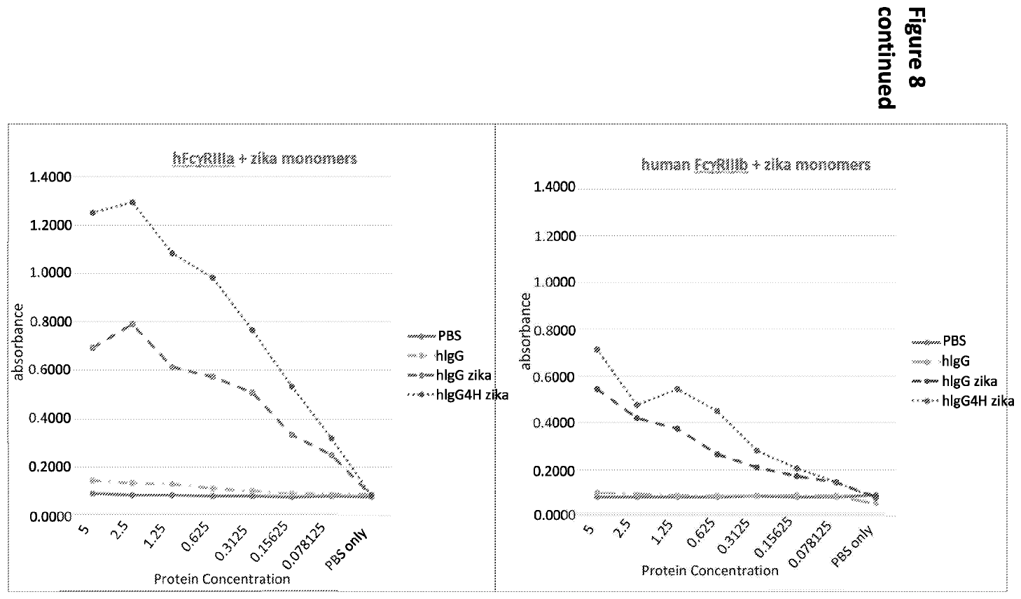

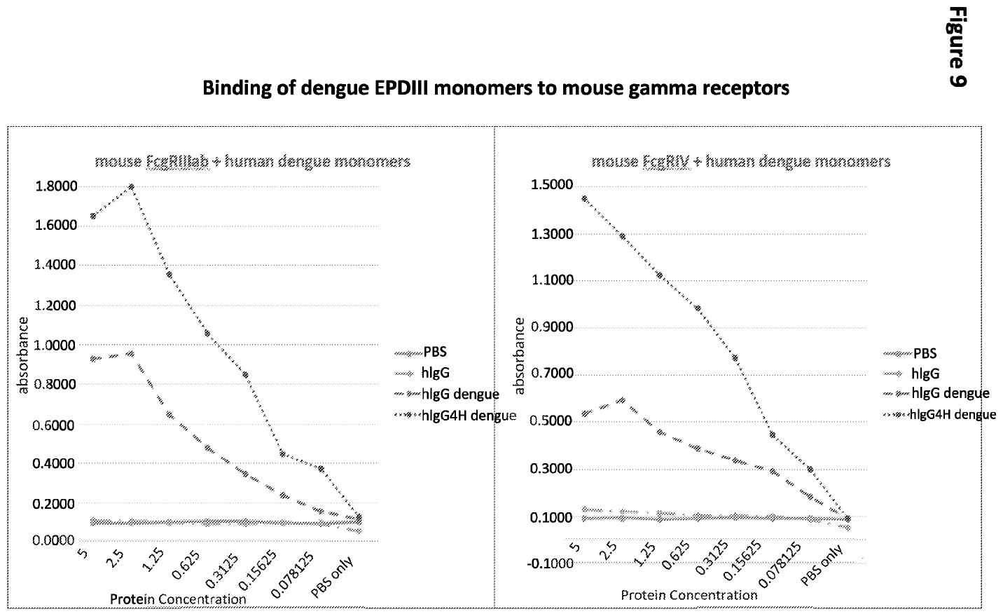

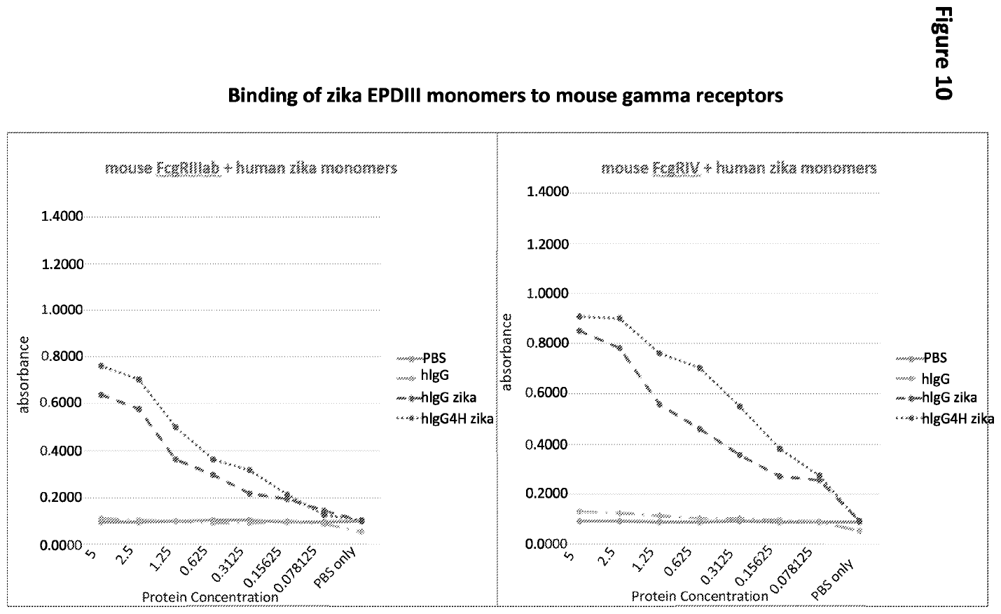

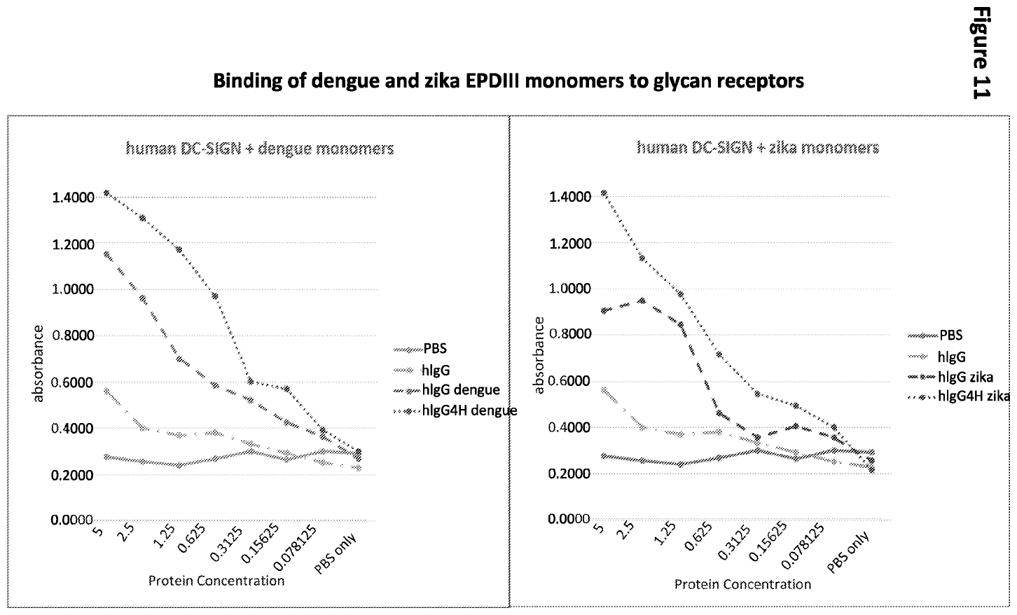

As shown in FIG. 3, the hexamer protein and the hexamer with a fused payload moiety do not bind or show minimal binding to FcγRIIa and FcγRIIIa receptors. However, upon introducing the hinge sequences into the constructs encoding the hexamer protein and the hexamer with a fused payload moiety, the resulting proteins (which maintain their hexameric structure) are able to bind to these receptors with much greater affinity. Additionally, FIG. 4 shows that the monomer protein (with hinge regions), and the monomeric protein (with hinge regions) and fused payload moiety binds FcγRIIa and FcγRIIIa receptors with greater avidity than comparator proteins without the hinge regions. FIG. 5 illustrates Western blot data which confirm the hexameric or monomeric structure of the Fc-fusion proteins comprising the hinge sequences. FIGS. 6 to 11 show the results of binding assays of the polypeptide of the invention with a fused payload moiety to various Fc-gamma and DC-SIGN receptors. As can been seen from the graphs in these figures, the presence of a plurality of hinge regions increases the binding of the polypeptide of the invention to the receptors, regardless of the type of payload moiety fused.

SEQUENCE INFORMATION
An exemplary hinge region which comprises
cysteine residues
SEQ ID NO: 1
DKTHTCPPCP
An exemplary hinge region which does not
comprise cysteine residues
SEQ ID NO: 2
DKTHTAPPAP
An exemplary fusion polypeptide of the
invention comprising 4 hinge regions
(underlined), three of which have been
modified to remove cysteines, and a
domain derived from the heavy chain
constant region of human IgG1
SEQ ID NO: 3
DKTHTAPPAPSRDKTHTAPPAPVDDKTHTAPPAPRSDKTHTCPPCPAPEL
LGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHN
HYTQKSLSLSPGK
An exemplary fusion polypeptide of the
invention comprising 4 hinge regions,
three of which have been modified to
remove cysteines, a domain derived from
the heavy chain constant region of human
IgG1, and a payload moiety underlined)
SEQ ID NO: 4
ATGVRAVPGNENSLEIEELARFAVDEHNKKENALLEFVRVVKAKEQMKNT
DFALATMYYLTLEAKDGGKKKLYEAKVWVKDVQWYGTLHNFKELQEFKPV
GDARSVDPWDKTHTAPPAPSRDKTHTAPPAPVDDKTHTAPPAPRSDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGK
An exemplary fusion polypeptide of the
invention comprising 4 hinge regions,
three of which have been modified to
remove cysteines, a domain derived from
the heavy chain constant region of human
IgG1, and an amino acid spacer and tail
(underlined)
SEQ ID NO: 5
DKTHTAPPAPSRDKTHTAPPAPVDDKTHTAPPAPRSDKTHTCPPCPAPEL
LGGPSVFLFPPKPKDTLMISRTPEVTCVVVDVSHEDPEVKFNWYVDGVEV
HNAKTKPREEQYNSTYRVVSVLTVCLQDWLNGKEYKCKVSNKALPAPIEK
TISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHN
HYTQKSLSLSPGKLVLGPPLYNVSLVMSDTAGTCY
An exemplary fusion polypeptide of the
invention comprising 4 hinge regions,
three of which have been modified to
remove cysteines, a domain derived from
the heavy chain constant region of human
IgG1, a payload moiety, a spacer, and a
tail
SEQ ID NO: 6
ATGVRAVPGNENSLEIEELARFAVDEHNKKENALLEFVRVVKAKEQMKNT
DFALATMYYLTLEAKDGGKKKLYEAKVWVKDVQWYGTLHNFKELQEFKPV
GDARSVDPWDKTHTAPPAPSRDKTHTAPPAPVDDKTHTAPPAPRSDKTHT
CPPCPAPELLGGPSVFLFPPKPKDTLMISRTPEVTCVVVDVSHEDPEVKF
NWYVDGVEVHNAKTKPREEQYNSTYRVVSVLTVCLQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPGKLVLGPPLYNVSLVMSDTAGTCY
An example of two hinge regions with a
short amino acid sequence between the
hinge regions.
SEQ ID NO: 7
DKTHTCPPCPRSDKTHTCPPCP
An example of three hinge regions with
a short amino acid sequence between
each of the hinge regions.
SEQ ID NO: 8
DKTHTCPPCPVDDKTHTCPPCPRSDKTHTCPPCP
An example of four hinge regions with a
short amino acid sequence between each
of the hinge regions.
SEQ ID NO: 9
DKTHTCPPCPSRDKTHTCPPCPVDDKTHTCPPCPRSDKTHTCPPCP
An example of two hinge regions with a
short amino acid sequence between the
hinge regions.
SEQ ID NO: 10
DKTHTAPPAPRSDKTHTCPPCP
An example of three hinge regions with a
short amino acid sequence between each
of the hinge regions.
SEQ ID NO: 11
DKTHTAPPAPVDDKTHTAPPAPRSDKTHTCPPCP
An example of four hinge regions with a
short amino acid sequence between each
of the hinge regions
SEQ ID NO: 12
DKTHTAPPAPSRDKTHTAPPAPVDDKTHTAPPAPRSDKTHTCPPCP
Spacer sequence
SEQ ID NO: 13
LVLG
Reference fusion polypeptide that forms
hexameric proteins (also designated
“Hexa-Fc or HexaGard”)
SEQ ID NO: 14
DKTHTCPPCP APELLGGPSV FLFPPKPKDT LMISRTPEVT
CVVVDVSHED PEVKFNWYVD GVEVHNAKTK PREEQYNSTY
RVVSVLTVCL QDWLNGKEYK CKVSNKALPA PIEKTISKAK
GQPREPQVYT LPPSREEMTK NQVSLTCLVK GFYPSDIAVE
WESNGQPENN YKTTPPVLDS DGSFFLYSKL TVDKSRWQQG
NVFSCSVMHE ALHNHYTQKS LSLSPGKLVL GPPLYNVSLV
MSDTAGTCY
DNA sequence of the amino acid sequence
set out by SEQ ID NO: 3
SEQ ID NO: 15
GACAAAACTCACACAGCCCCACCGGCCCCATCTAGAGACAAAACTCACAC
AGCCCCACCGGCCCCAGTCGACGACAAAACTCACACAGCCCCACCGGCCC
CAAGATCTGACAAAACTCACACATGCCCACCGTGCCCAGCACCTGAACTC
CTGGGGGGACCGTCAGTCTTCCTCTTCCCCCCAAAACCCAAGGACACCCT
CATGATCTCCCGGACCCCTGAGGTCACATGCGTGGTGGTGGACGTGAGCC
ACGAAGACCCTGAGGTCAAGTTCAACTGGTACGTGGACGGCGTGGAGGTG
CATAATGCCAAGACAAAGCCGCGGGAGGAGCAGTACAACAGCACGTACCG
TGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCT
GCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGC
AGCAGGGGAACGTCTTCTCATGCTCCGTGATGCACGAGGCTCTGCACAAC
CACTACACGCAGAAGAGCCTCTCCCTGTCTCCGGGTAAATGA
DNA sequence of the amino acid sequence
set out by SEQ ID NO: 5
SEQ ID NO: 16
GACAAAACTCACACAGCCCCACCGGCCCCATCTAGAGACAAAACTCACAC
AGCCCCACCGGCCCCAGTCGACGACAAAACTCACACAGCCCCACCGGCCC
CAAGATCTGACAAAACTCACACATGCCCACCGTGCCCAGCACCTGAACTC
CTGGGGGGACCGTCAGTCTTCCTCTTCCCCCCAAAACCCAAGGACACCCT
CATGATCTCCCGGACCCCTGAGGTCACATGCGTGGTGGTGGACGTGAGCC
ACGAAGACCCTGAGGTCAAGTTCAACTGGTACGTGGACGGCGTGGAGGTG
CATAATGCCAAGACAAAGCCGCGGGAGGAGCAGTACAACAGCACGTACCG
TGTGGTCAGCGTCCTCACCGTCTGCCTCCAGGACTGGCTGAATGGCAAGG
AGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCT
GCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGC
AGCAGGGGAACGTCTTCTCATGCTCCGTGATGCACGAGGCTCTGCACAAC
CACTACACGCAGAAGAGCCTCTCCCTGTCTCCGGGTAAATTAGTCCTAGG
ACCCCCCCTGTACAACGTGTCCCTGGTCATGTCCGACACAGCTGGCACCT
GCTACTGA
Exemplary payload sequence-Zika virus
EPIII domain
SEQ ID NO: 17
GMSYSMCTGKFKIVKEIAETQHGTIVIRVQYEGDGSPCKIPFEIMDLEKR
HVLGRLITVNPIVTEKDSPVNIEAEPPFGDSYIIIGVEPGQLKLNWFKKG
SSIGQ
Exemplary payload sequence-Dengue virus
EPIII domain
SEQ ID NO: 18
GMSYSMCTGKFKIVKEIAETQHGTIVIRVQYEGDGSPCKIPFEIMDLEKR
HVLGRLITVNPIVTEKDSPVNIEAEPPFGDSYIIIGVEPGQLKLNWFKKG
SSIGQ
Exemplary payload sequence-F1 adhiron
SEQ ID NO: 19
ATGVRAVPGNENSLEIEELARFAVDEHNKKENALLEFVRVVKAKEQMKNT
DFALATMYYLTLEAKDGGKKKLYEAKVWVKDVQWYGTLHNFKELQEFKPV
GDA

1. A fusion polypeptide comprising
a domain derived from an immunoglobulin heavy chain constant region; and
a plurality of hinge regions derived from IgG hinge sequences.

2-46. (canceled)
